(19) United States
(12) Patent Application Publication
(10) Pub. No.: US 2008/0076703 A1
(43) Pub. Date: Mar. 27, 2008

(54) Compositions and methods for the enhanced uptake of therapeutic agents through the bladder epithelium

(75) Inventors: Bahram Memarzadeh, San Carlos, CA (US); Nagarajan Ramesh, Sunnyvale, CA (US); David Frey, Half Moon Bay, CA (US); DeChao Yu, Palo Alto, CA (US)

Correspondence Address: ROPES & GRAY LLP;PATENT DOCKETING 39/361, 1211 AVENUE OF THE AMERICAS, NEW YORK, NY, 10036-8704, US

(73) Assignee: Cell Genesys, Inc., South San Francisco, CA (US)

(21) Appl. No.: 11/891,659

(22) Filed: Aug. 9, 2007

Related U.S. Application Data
(63) Continuation of application No. 10/885,309, filed on Jul. 7, 2004, now abandoned.
(60) Provisional application No. 60/485,115, filed on Jul. 8, 2003.

(51) Int. Cl. A61K 47/08; A61K 31/337; A61P 13/10; A61P 35/00; A61K 38/02

(52) U.S. Cl. 514/2; 514/449; 514/777

(57) ABSTRACT

Compositions and methods for enhancing introduction of therapeutic agents into the bladder epithelium for the treatment of bladder diseases and disorders such as bladder cancer are described. According to one method, the luminal surface of the bladder is contacted with a composition comprising a bladder enhancer and a therapeutic agent for the treatment of the bladder disease. According to an alternative method, the luminal surface of the bladder is first contacted with a pretreatment composition comprising a bladder enhancer and subsequently contacted with a composition comprising a therapeutic agent for the treatment of the bladder disease. The transduction enhancing agent can be a mono-, di-, or poly-saccharide having a lipophilic substituent such as n-dodecyl-β-D-maltoside (DDM). Compositions comprising a transduction enhancing agent and a therapeutic agent for the treatment of a bladder disease are also described.

This application claims priority from U.S. Provisional Application Ser. No. 60/485,115 filed Jul. 8, 2003. The entirety of that provisional application is incorporated herein by reference.

This application is also related to U.S. patent application Ser. No. 10/327,869, filed Dec. 26, 2002 and U.S. patent application Ser. No. 10/743,813, filed Dec. 24, 2003, both of which are incorporated herein by reference in their entirety.

### BACKGROUND

1. Technical Field

The present invention relates generally to enhanced uptake of therapeutic agents for the treatment of bladder disease, and, in particular, to agents and methods that allow therapeutic agents for the treatment of the bladder to pass through the bladder epithelium.

2. Background of the Technology

Bladder cancer is a commonly occurring cancer and more than 50,000 new cases are diagnosed every year [Jemal, A., et al., (2002), CA Cancer J Clin, 52: 23-47]. Bladder cancer is a superficial disease confined to the mucosa in the majority of patients. Of the various therapeutic modalities available, transurethral resectioning of the tumor is considered to be the most effective treatment for the management of superficial bladder cancer. However, 70% of these superficial bladder tumors will recur after endoscopic resectioning, and 20% progress to life-threatening invasive diseases within 2 years of cystectomy. See Raghavan, et al., N. Engl. J. Med., 322, 16, 1129-1138 (1990).

More than 90% of bladder tumors are transitional cell carcinomas (TCC) [Soloway, M. S et al., (2002), J. Urol. 167: 1573-1583]. Risk factors for the development of TCC include cigarette smoking and exposure to arylamines (such as aniline dyes), certain drugs and pelvic radiation. Urothelial tumors fall into two major groups—superficial and invasive, with substantially different natural histories. Approximately 80% of superficial tumors remain confined to the mucosa and submucosa through most of their natural history, whereas most invasive tumors exhibit their invasive property at initial presentation and are then associated with a worse prognosis and propensity to metastasize. Unfortunately, bladder cancer often recurs despite repeated intravesical instillation of therapeutic agents. Many superficial bladder tumors are relatively well differentiated and do not have a high rate of proliferation and local delivery into the bladder should avoid the difficulties of systemic administration of therapeutic agents. New therapeutic modalities are needed to augment or replace the existing options in the management of urological cancers.

The mammalian bladder maintains high electrochemical gradients between urine and blood, permitting the kidney to modify body chemistries through urinary excretion [Zeidel, M. L. (1996) et al., Am J Physiol Renal Fluid Electrolyte Physiol. 271: F243-F245].

To perform this function, the urothelium maintains a tight permeability barrier. The permeability barrier consists of three components: the apical substances, tight junctions, and an active trafficking mechanism that moves membrane from subapical vesicles into the apical membrane in response to stretching or filling of the bladder [Negrete, H. O., et al., (1996) et al., Am J Physiol Renal Fluid Electrolyte Physiol. 271: F886-F894]. In addition the bladder surface is lined with a sulfated polysaccharide glycosaminoglycan (GAG), which acts as a nonspecific anti-adherence factor and possibly as a first line anti-infection defense mechanism [Parson, C. L. et al., (1990), J Urol. 143: 139-142]. Intravesical chemotherapy is used in combination with transurethral resection for treatment and prophylaxis of recurrent and/or multifocal superficial bladder cancer [Melekos, M. D. and Moutzouris, G. D. (2000). Curr Pharm Des. 6: 345-359]. Compared to patients treated by surgery alone, those receiving adjuvant intravesical chemotherapy have a reduced tumor recurrence rate. The target cells for intravesical chemotherapy may be present in the bladder cavity and at various tissue layers in the bladder wall.

Patients and animal studies in several laboratories have showed that the variability in urine pharmacokinetics, drug delivery to the tumor tissue and tumor chemo sensitivity may contribute to the varying patient responses [Dalton, J. T., et al, (1991) Cancer Res. 51: 5144-5152]. The ease of drug penetration in the superficial and deeper tissues and the drug concentrations at various tissue depths in the bladder wall are therefore important determinants of treatment effectiveness.

The rationale for intravesical therapy is to expose tumor cells located in the bladder wall to concentrations of therapeutic agents at much higher than those achieved systemically during treatment. Target site drug exposure is an important determinant of treatment efficacy, while systemic exposure is an important determinant of host toxicity.

The exposure of tumor cells located in the bladder wall to a given therapeutic agent is highly dependent on the total amount of drug that enters the bladder following intravesical instillation. The total amount of drug that enters the bladder following intravesical instillation provides clinicians with an approximate estimate of the total drug that is taken up by tumor cless located in the bladder wall, and the kinetics of drug penetration through the bladder wall. For example, the bladder exposure to intravesically-instilled mitomycin C demonstrated large intra and intersubject variability [Dalton, J. T et al., (1991) Cancer Res. 51: 5144-5152]. It was postulated that the residual urine present in the bladder at the time of instillation, the production of urine during therapy and drug removal by absorption and degradation during therapy were the major determinants of the target site exposure to mitomycin C. Concurrent studies demonstrated that the total amount of drug that enters the bladder following intravesical instillation is an important determinant of therapeutic efficacy in the urothelium, lamina propria and muscle layers.

In vivo studies have demonstrated that various agents (e.g., acetone, DMSO, protamine sulfate) can break down the protective “mucin” layer that protects the bladder epithelium from bacteria, viruses and other pathogens. See, for example, Monson, et al., J. Urol., 145, 842-845 (1992) and Parsons, et al., J. Urol., 143, 139-142 (1990). Methods of modifying the bladder surface to enhance gene transfer have also been disclosed [Siemens, et al., J. of Urology, 165, 667-671 (2001)].

There remains a need for improved methods of delivering therapeutic agents for the treatment of bladder disorders by way of the bladder epithelium.

### SUMMARY OF THE INVENTION

According to a first aspect of the invention, a method for treating a bladder disorder such as bladder cancer is provided. According to this aspect of the invention, the method involves contacting the luminal surface of the bladder with a pretreatment composition comprising a bladder uptake enhancing agent; and subsequently contacting the luminal surface of the bladder with a composition comprising a therapeutic agent for the treatment of the bladder disorder. According to this aspect of the invention, the bladder uptake enhancing agent is a mono-, di-, or poly-saccharide having a lipophilic substituent. Exemplary bladder uptake enhancing agents include compounds having the following general formula (I) or the following general formula (II): 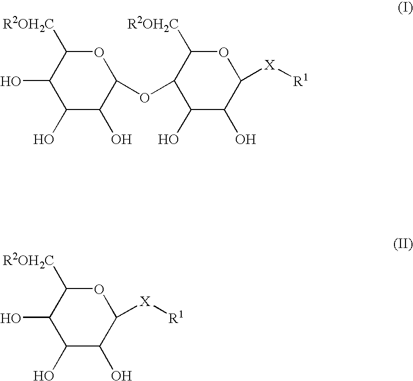
wherein X is a sulfur or oxygen atom, R¹ is an alkyl group and each R² is independently hydrogen or a moiety represented by: 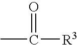
wherein R³ is an alkyl group. The pretreatment composition can further include an oxidizing agent. The therapeutic agent can be a chemotherapeutic agent, a cytokine, a synthetic small molecule drug, a natural product, a radionuclide, or a protein.

According to another aspect of the invention, a method of treating a bladder disorder, such as cancer of the bladder, is provided which includes contacting the luminal surface of the bladder with a pretreatment composition comprising a bladder uptake enhancing agent having a structure represented by the following general formula (III): 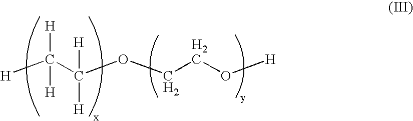
wherein x and y are positive integers; and subsequently contacting the luminal surface of the bladder with a composition comprising a therapeutic agent for the treatment of the bladder disorder. According to a preferred embodiment, x in the above formula (III) is 6 and y in the above formula (III) is 8-10 and the pretreatment composition comprises about 0.02 to about 0.05 wt. % of the bladder uptake enhancing agent. The therapeutic agent can be a chemotherapeutic agent, a cytokine, a synthetic small molecule drug, a natural product, a radionuclide, or a protein.

According to a further aspect of the invention, a method of treating a bladder disorder such as cancer of the bladder is provided which includes contacting the luminal surface of the bladder with a pretreatment composition comprising a bladder uptake enhancing agent having a structure represented by the following general formula (IV) or the following general formula (V): 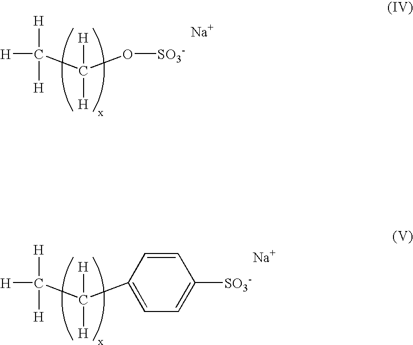
wherein x is a positive integer and subsequently contacting the luminal surface of the bladder with a composition comprising a therapeutic agent for the treatment of the bladder disorder. The therapeutic agent can be a chemotherapeutic agent, a cytokine, a synthetic small molecule drug, a natural product, a radionuclide, or a protein.

According to yet a further aspect of the invention, a composition comprising a bladder uptake enhancing agent and a therapeutic agent for the treatment of a bladder disorder such as cancer of the bladder is provided. According to this aspect of the invention, the bladder uptake enhancing agent is a mono-, di-, or poly-saccharide having a lipophilic substituent. Exemplary bladder uptake enhancing agents include compounds having the following general formula (I) or the following general formula (II): 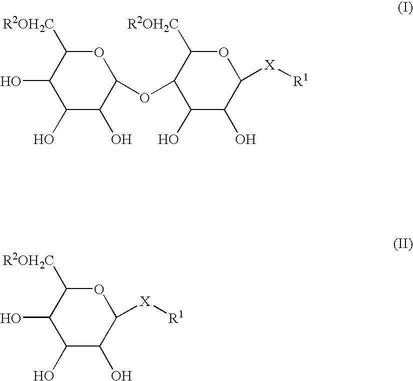
wherein X is a sulfur or oxygen atom, R¹ is an alkyl group and each R² is independently hydrogen or a moiety represented by: 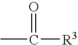
wherein R³ is an alkyl group. The therapeutic agent can be a chemotherapeutic agent, a cytokine, a synthetic small molecule drug, a natural product, a radionuclide, or a protein. A method for treating bladder disorders such as cancer of the bladder comprising contacting the luminal surface of the bladder with a composition as set forth above is also provided.

In one preferred aspect of the invention, the bladder pretreatment agent is n-dodecyl-β-D-maltoside (DDM), a non-ionic organic surfactant, which overcomes the permeability barrier imposed by the sulfated polysaccharide glycosaminoglycan (GAG) which lines the bladder surface and acts as a nonspecific anti-adherence factor.

According to another preferred aspect of the invention, a composition comprising n-dodecyl-β-D-maltoside (DDM), and a therapeutic agent for the treatment of a bladder disorder is provided. The therapeutic agent can be a chemotherapeutic agent, a cytokine, a synthetic small molecule drug, a natural product, a radionuclide, or a protein. A method for treating a bladder disorder such as cancer of the bladder comprising contacting the luminal surface of the bladder with a composition as set forth above is also provided.

Many therapeutic and pharmaceutical substances, approved or at the research stage, lack good water solubility in water. The present invention provides a means for such therapeutic and pharmaceutical substances to be delivered through the GAG layer of the bladder thereby enhancing therapeutic efficacy. Such delivery may be local to the bladder for the treatment of bladder disease or may effect systemic distribution of the therapeutic or pharmaceutical substance following bladder instillation.

### BRIEF DESCRIPTION OF THE DRAWINGS

FIGS. 1A-1F illustrate uptake of paclitaxel following intravesical instillation in murine bladders and histological examination of the bladder under a fluorescence microscope. The bladders were pretreated either with PBS (FIGS. 1A-1C) or DDM (FIGS. 1D-1F). Nuclei were stained and visualized following DAPI staining at a magnification of 10×.

FIGS. 2A-2F illustrate TAT-peptide uptake following intravesical instillation in murine bladders and histological examination of the bladder under a fluorescence microscope. The bladders were pretreated either with PBS (FIGS. 2A-2C) or DDM (FIGS. 2D-2F). Nuclei were stained and visualized following DAPI staining at a magnification of 10×.

### DETAILED DESCRIPTION OF PREFERRED EMBODIMENTS

Methods which involve the use of bladder uptake enhancing agents to render the bladder umbrella cell or GAG layer more susceptible to treatment with a therapeutic agent than it would be without treatment are provided. A “bladder uptake enhancing agent” as used herein refers to a compound which facilitates uptake of a therapeutic agent through the bladder epithelium. In the presence of a “bladder uptake enhancing agent” of the invention, the therapeutic agent is able to penetrate the bladder umbrella cell or GAG layer such that the therapeutic agent gains greater access to cells beyond the luminal surface of the bladder.

Exemplary bladder uptake enhancing agents include: dodecyl surfactants; dodecylmaltosides; dodecyl alcohol polyoxyethylene ethers (i.e., polidocanol); and sodium dodecylbenzenesulphonic acid/hypochlorous acid complex (i.e., oxychlorosene).

According to one embodiment, the luminal surface of the bladder can be treated with a composition comprising a bladder uptake enhancing agent prior to contacting the luminal surface of the bladder with a therapeutic agent for the treatment of a bladder disorder such as bladder cancer. The therapeutic agent for the treatment of the bladder can be a chemotherapeutic agent, a cytokine, a synthetic small molecule drug, a natural product, a radionuclide, or a protein.

In addition to pre-treatment of the bladder surface with the bladder uptake enhancing agent, methods including the co-administration of the therapeutic agent for the treatment of the bladder and the bladder uptake enhancing agent to the bladder are also provided. Accordingly, co-formulations of any one of the bladder uptake enhancing agents and any one or more of the therapeutic agents for the treatment of bladder disorders are also provided.

In one aspect, the invention provides for use of bladder uptake enhancing agents to facilitate systemic distribution of a therapeutic agent for treatment of disease outside the bladder.

Reagents which can be used to enhance bladder uptake in the bladder epithelium can be grouped as either single compounds or as mixed reagents (i.e., mixtures of compounds). Exemplary single compounds include non-ionic surfactants, alcohols, polymers and ionic surfactants. Exemplary surfactants include: Poloxamer 407 (Pluronic® 127); poloxamer 188 (Pluronic® F68); Polidocanol; n-dodecyl-β-D-glucopyranoside (which can also be classified as a sugar-based surfactant); n-dodecyl-β-D-maltoside (which can also be classified as a sugar-based surfactant); Tween® 20; Triton® X-100; Forlan® C-24 (PEG Cholesterol); decyl-β-D-maltoside (which can also be classified as a sugar-based surfactant); 6-cyclohexylhexyl-β-D-maltoside (which can also be classified as a sugar-based surfactant); and sodium tetradecyl sulfate (e.g., Tromboject®). Exemplary alcohols that can be used to enhance uptake by the bladder epithelium according to the invention include benzyl alcohol and ethanol. Exemplary polymers that can be used to enhance uptake by the bladder epithelium according to the invention include HPMC 2910, PVA, and poly-lysine. Exemplary ionic surfactants that can be used to enhance uptake by the bladder epithelium according to the invention include: DC-Chol [Cholesteryl 3β-N-(dimethylaminoethyl)carbamate]; the sodium salt of dodecyl benzenesulfonic acid; and sodium dodecyl sulfate. Exemplary mixed reagents that can be used to enhance uptake by the bladder epithelium according to the invention include: In vivo GeneSHUTTLE™ (a reagent comprising DOTAP+Cholesterol available from Qbiogene of Carlsbad, Calif.) and oxychlorosene (i.e., sodium dodecylbenzenesulphonic acid/hypochlorous acid complex).

Sugar Based Surfactants (Saccharides)

The bladder uptake enhancing agent according to the invention can be a sugar (e.g., a mono-, di-, or poly-saccharide) having a lipophilic substituent. In particular, the bladder uptake enhancing agent according to the invention can be any mono-, di-, or poly-saccharide having a lipophilic substituent. According to a preferred embodiment of the invention, the bladder uptake enhancing agent is a di-saccharide having a lipophilic substituent. Exemplary di-saccharides include maltose or sucrose. Other di-saccharides having lipophilic substituents, however, can also be used including lactose, isomaltose, trehalose or cellobiose.

The lipophilic substituent can be linear (e.g., a straight chain n-alkane or alkene) or non-linear (e.g., cyclic or branched chain alkanes or alkenes). The lipophilic substituent can also be an alkanoic acid residue. The length of the lipophilic substituent can be varied to achieve the desired hydrophilic-lipophilic balance. Exemplary sugar based surfactants include n-dodecyl-β-D-maltoside, 6-cyclohexylhexyl-β-D-maltoside and n-decyl-β-D-maltoside. The chemical formula for n-dodecyl-β-D-maltoside and n-decyl-β-D-maltoside is given below: 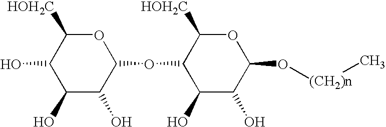
where n is 11 and 9, respectively. The chemical formula for 6-cyclohexylhexyl-β-D-maltoside is: 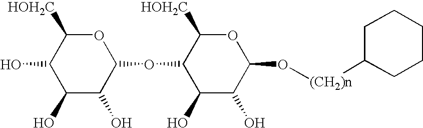
where n is 6.

Compounds in this class of surfactants having a shorter hydrophilic moiety such as n-dodecyl-β-D-glucopyranoside can also be used according to the invention. The chemical formula for n-dodecyl-β-D-glucopyranoside is: 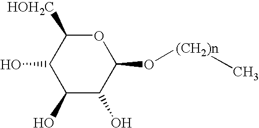
where n is 11. The relative sizes of the hydrophilic and lipophilic portions of the molecule may influence the degree of bladder uptake enhancement. Therefore, shorter chain n-alkyl-β-D-glucopyranosides (e.g., n-hexyl-β-D-glucopyranoside) may exhibit improved uptake through the bladder epithelium.

Any mono-, di-, or poly-saccharide having a lipophilic substituent can be used as a bladder uptake enhancing agent according to the invention. Exemplary di-saccharide compounds include sucrose, lactose, maltose, isomaltose, trehalose, and cellobiose. The lipophilic substituent preferably comprises an alkyl or alkenyl group. According to a preferred embodiment of the invention, the lipophilic substituent is an alkanoic acid residue.

Although the β-forms of the mono- and di-saccharides are described above, the α-forms of these and other mono-, di-, or poly-saccharide compounds can also be used according to the invention. Exemplary α-saccharide bladder uptake enhancing agents according to the invention include n-dodecyl-α-D-maltoside, n-hexyl-α-D-glucopyranoside and 6-cyclohexylhexyl-α-D-maltoside. Additionally, either the D- or L-forms of the mono-, di-, or poly-saccharides may be used as bladder uptake enhancing agents according to the invention.

The examples provided herein demonstrate that pretreatment of the bladder with a 0.1% solution of n-dodecyl-β-D-maltoside (DDM) for 5 min permitted efficient access to the normal urothelium as well as to the orthotopic bladder tumors present on the luminal surface of the bladder. Electron Microscopy of the DDM pretreated bladder showed that the distinct GAG layer was absent on the urothelium of DDM-pretreated bladders and had been replaced by a spongy, non-contiguous and weakly stained GAG layer. Disruption of tight junctions was also seen along the surface in the DDM-pretreated bladder. U.S. patent application Ser. Nos. 10/327,869 and 10/743,813 (expressly incorporated by reference herein) illustrate that adenovirus particles with a size range of 70 nm to 100 nm have been able to penetrate the bladder tissue after DDM pretreatment. According to the present invention, micronized or nano-particles of pharmaceuticals or therapeutic agents with poor aqueous solubility may be suspended in aqueous medium and instilled in DDM-treated bladder. These small particles enter the openings provided by DDM treatment and thereby reside in bladder tissue for long periods of time. The active compound from these particles may therefore slowly penetrate the tissue and provide therapeutic efficacy for the treatment of bladder disease.

Since DDM pretreatment leads to transient breaks in the barrier functions of the GAG and epithelial plaques, such pretreatment allows for increased uptake of salts, chemo or other therapeutic agents, peptides, proteins etc. by the bladder epithelium. The examples which follow describe the results of studies on bladder tissue uptake of a chemotherapeutic agent (Oregon Green 488 Paclitaxel; Example 2), a membrane permeation peptide (FITC-LC-TAT peptide; Example 3) and systemic uptake and availability of a salt solution (Sodium Fluorescein; Example 4) instilled intravesically in the bladder following retention for a short time period.

Ionic Alkyl Surfactants

Ionic alkyl surfactants can also be used as bladder uptake enhancing compounds according to the invention. Exemplary ionic alkyl surfactants include sodium dodecyl sulfate which has a formula represented by:

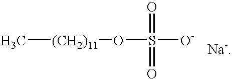

Another exemplary ionic surfactant is the sodium salt of dodecyl-benzenesulfonic acid which has a chemical formula represented by:

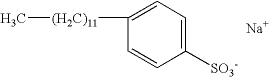

The ionic alkyl surfactants consist of two portions, a hydrophilic portion and a lipophilic portion. The arrangement of these portions of the molecule is similar to the sugar-based enhancing agents described above. According to the invention, compounds similar to those set forth above and having variations in alkyl substitution can also be used.

Alkyl(ether) Alcohols

An alkyl ether compound can also be used as bladder uptake enhancing compound according to the invention. Exemplary alkyl ether compounds include polidocanol, which has the following chemical formula:
C₁₂H₂₆—O—(CH₂—CH₂—O)_{˜9}
and a total formula of ˜C₃₀H₆₂O₁₀. Polidocanol is available commercially under the name Thesit®, which is a registered trademark of Desitin-Werk, Carl Klinke GmbH, Hamburg, Germany. There are several other chemical names for polidocanol such as polyethyleneglycoldodecyl ether [9002-92-0], lauryl alcohol, and macrogol lauryl ether.

Another exemplary alkyl ether compound that can also be used as a bladder uptake enhancing compound according to the invention is alpha-[4-(1,1,3,3-tetramethylbutyl)phenyl]-omega-hydroxypoly(oxy-1,2-ethanediyl) (CAS No. 9002-93-1) which is sold under the name Triton® X-100. This compound has the general formula: 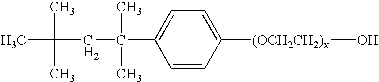
wherein x=10. A similar compound having a cyclohexane ring rather than a benzene ring can also be used as a transduction enhancing agent according to the invention. This compound has the following chemical structure: 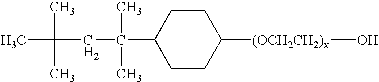
wherein x=10. Compounds of the above type wherein x is any positive integer can also be used as bladder uptake enhancing compounds according to the invention. Similar alkyl(ether) compounds having the general structure of: 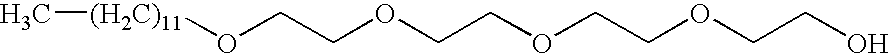
are also commercially available and can be used as bladder uptake enhancing compounds according to the invention. The trade name for these compounds is “Brij”. The compound shown above is designated “Brij 56”. Brij 56 has the chemical formula C₂₀H₄₂O₅. Another commercially available compound, “Brij 58”, has the chemical formula C₅₆H₁₁₄O₂₁. Any of the above mentioned alkyl(ether) compounds can be used as bladder uptake enhancing agents according to the invention.
Sodium Oxychlorosene

A composition comprising a sodium salt of dodecylbenzenesulfonic acid and hypochlorous acid (i.e., sodium oxychlorosene) at a pH of about 6.5 to 6.9 can also be used as a bladder uptake enhancing agent according to the invention. Sodium oxychlorosene is available under the name Clorpactin WCS-90 (manufactured by Guardian Labs and sold by Cardinal Health). Sodium oxychlorosene has been used to treat urinary tract infections and in abdominal and plastic surgery.

Polymers with Alternating Hydrophilic and Lipophilic Units

Polymeric compounds comprising repeating sequences of alternating or identical monomers were can also be used as transduction enhancing agents according to the invention. Exemplary compounds of this type include Poloxamer 407 (Pluronic 127) having a structure represented by the following formula: 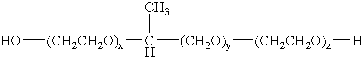
Poloxamer polymers are available in a wide range of HLB values.
Additional Bladder Uptake Enhancing Compounds

Additional compounds can also be used as bladder uptake enhancing agents according to the invention. These compounds include ω-undecylenyl-β-D-maltopyranoside, which has a structure represented by: 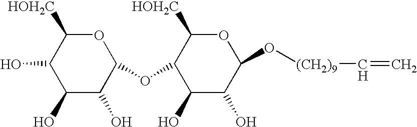
Sugar based thiolic compounds such as alkyl-β-D-thioglucopyranosides having a general structure represented by: 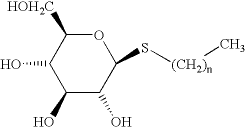
may also be employed.

Additionally, alkyl-β-D-thiomaltopyranosides having a general structure represented by: 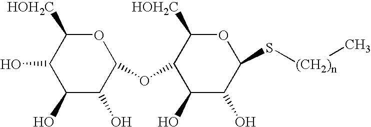
may also be used as bladder uptake enhancing compounds according to the invention.

Additionally, compounds having a positive charge such as 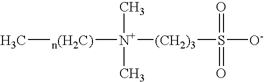
can also be used as bladder uptake enhancing compounds.

Additionally, compounds wherein the lipophilic and hydrophilic parts are connected via a carboxylic bond can also be employed as tranduction enhancing agents. An exemplary compound of this type is 6-O-methyl-n-heptylcarboxyl-α-D-glucopyranoside which is represented by the following formula:

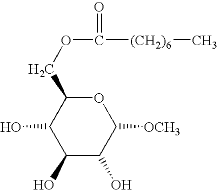

Sugar based compounds having alkyl groups with side groups or other modifications may also be used. Exemplary compounds of this type include 2-propyl-1-pentyl-β-D-maltopyranoside having a structure represented by:

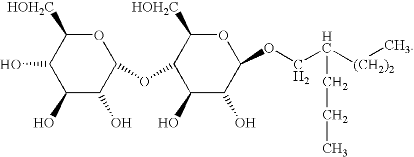

Sarcosine compounds may also be used as bladder uptake enhancing agents according to the invention. Exemplary sarcosine compounds include sodium alkyl sarcosine having a structure represented by:

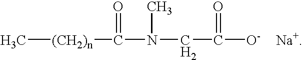

Various substituted sugars can also be used as bladder uptake enhancing compounds. An exemplary substituted sugar which can be used as a bladder uptake enhancing compound is a sucrose mono alkyl ester having a chemical structure represented by: 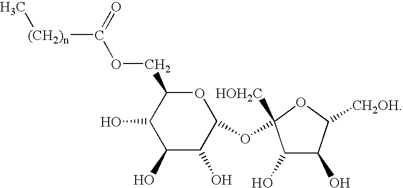
Exemplary compounds of this type include compounds wherein n=10 (i.e., sucrose monolaurate).

Methods of treating the luminal surface of the bladder are also provided. According to a preferred embodiment of the invention, the bladder is treated by instillation using bladder catheterization. According to this embodiment of the invention, any urine in the bladder is first removed and the bladder is optionally washed with a buffer (e.g., PBS). A composition comprising the bladder uptake enhancing agent is then applied to the luminal surface of the bladder (e.g., by instillation). The transduction enhancing solution may be incubated for some specified time or drained immediately. Multiple treatments with the composition comprising the bladder uptake enhancing agent can be performed. After treatment with the bladder uptake enhancing agent, the luminal surface of the bladder may be washed with a buffer (e.g., PBS). A solution comprising the therapeutic agent for the treatment of a bladder disorder can then be introduced into the bladder (e.g., by instillation). The solution comprising the therapeutic can be removed immediately or, alternatively, the solution can be allowed to incubate for a certain amount of time. After treatment, the bladder surface can again be washed with a buffer solution (e.g., PBS). According to a preferred embodiment of the invention, about 50 to about 500 ml of the bladder uptake enhancing composition is delivered to the bladder by instillation for each treatment.

Alternatively, a composition comprising the bladder uptake enhancing agent and the therapeutic agent can be used to treat the luminal bladder surface. According to this embodiment of the invention, any urine in the bladder is first removed and the bladder is then optionally washed with a buffer (e.g., PBS). A composition comprising the bladder uptake enhancing agent and the therapeutic agent is then applied to the luminal surface of the bladder. The solution may be incubated for some specified time or drained immediately. After treatment, the luminal surface of the bladder may again be washed with a buffer (e.g., PBS).

Although phosphate buffered saline (PBS) is the preferred buffer, any other pharmaceutical buffer can be used according to the invention. Exemplary buffers include sodium phosphate/sodium sulfate, Tris buffer, glycine buffer, sterile water and other buffers known in the art, including those described by Good, et al., Biochemistry 5, 467 (1966). The pH of the buffer used can be in the range of 6.4 to 8.4, preferably 7 to 7.5, and most preferably 7.2 to 7.4.

The composition comprising the bladder uptake enhancing agent according to the invention can also comprise an oxidizing agent. Exemplary oxidizing agents include, but are not limited to, chlorite compounds, hypochlorous acid, hydrogen peroxide, and peroxyacetic acid. According to a preferred embodiment of the invention, any of the single compound bladder uptake enhancing agents can be combined with an oxidizing agent and the resulting composition used as a bladder uptake enhancing agent.

According to exemplary embodiments of the invention, a 0.1% or 0.2% oxychlorosene solution can be used to treat the luminal bladder surface. Treatment at any of these concentrations can be conducted for a period of 5 minutes. According to further exemplary embodiments of the invention, a 0.02%, 0.05% or 0.2% polidocanol solution can be used to pre-treat the luminal bladder surface. Treatment at any of these concentrations can be conducted for a period of 5 minutes. According to further exemplary embodiments of the invention, a 0.02%, 0.05%, or 0.2% n-dodecyl-β-D-maltoside solution can be used to treat the luminal bladder surface. Treatment at any of these concentrations can be conducted for a period of 5 minutes. Alternatively, a 0.2% solution of the sodium salt of dodecyl benzenesulfonic acid can be used to pre-treat the luminal bladder surface. Treatment can be conducted for a period of 5 minutes. All percentages given above are weight percentages.

Solutions having the following concentrations of bladder uptake enhancing agents can be used as a pretreatment of the luminal bladder surface according to the invention: 0.02%-0.5% polidocanol; 0.02-0.5% n-dodecyl-β-D-maltoside; 0.1% 6-cyclohexylhexyl-β-D-maltoside; 0.1%-0.4% oxychlorosene; 0.2% sodium salt of dodecyl benzenesulfonic acid; 0.1% sodium dodecyl sulphate; 0.1% decyl-β-D-maltoside and 0.1% Triton® X-100. All percentages given above are weight percentages. The pretreatment solution can be contacted with the luminal bladder surface for up to 15 minutes or more. For example, the pretreatment solution can be contacted with the luminal bladder surface for 5 to 15 minutes.

Exemplary therapeutic agents according to the invention include, but are not limited to, chemotherapeutic agents, cytokines, synthetic small molecule drugs, natural products, radionuclides and polypeptides (e.g., proteins). Exemplary chemotherapeutic agents include, but are not limited to, taxane (docetaxel), doxorubicin, mitomycin C, valrubicin, epirubicin, thiotepa, interferon alpha and other cytokines therapeutic activities.

Other exemplary therapeutic agents for the treatment of bladder or other disorders include, but are not limited to, antisense Bcl-2 oligonucleotide; EGF-dextran-Tc (radionuclide to EGF-receptor); BCG; folic acid analogs; methotrexate (MTX); pyrimidine analogs; fluorouracil (5-FU); fluorodeoxyuridine; Cytarabine; purine analogs such as 6-mercaptopurine (6-MP) and 6-thioguanine (6-TG); alkylating agents such as nitrogen mustards, mechlorethamine, cyclophosphamide (Cytoxan®), Melphalan and Chlorambucil; natural products, such as vinca alkaloids, vincristine (Oncovin®), vinblastine (Velban®), vinorelbine (Navelbine®), epipodophylotoxins, etoposide (VePesid®, VP-16), taxol (Paclitaxel®), antiumor antibiotics, anthracyclines including doxorubicin hydrochloride (Adriamycin®), daunorubicin, idarubicin, mitoxantrone (an Anthracenedione that lacks a sugar moiety), Bleomycin (Blenoxane®), Dactinomycin (actinomycin D), Mitomycin C, Plycamycin (Mithramycin).

Various miscellaneous agents can also be used as therapeutic agents for the treatment of bladder or other disorders according to further embodiments of the invention. Exemplary miscellaneous agents include Cisplatin, Carboplatin, Asparaginase, hydroxyurea, Mitotane (o,p′-DDD; Lysodren), Anti-Estrogen (tamoxifen citrate), Corticosteroid (Prednisone); or Mebendazole (also referred to as Mebendozole).

Mebendazole (which is a benzimidazole) is an anti-parasitic medication which has been suggested for use as an anti-cancer agent. Mebendazole binds to tubulin and interferes with microtubule formation. Mebendazole has poor bio-availability so systemic actions are minimal. Mebendazole and other benzimidazoles can also be used as therapeutic agents for the treatment of bladder disorders according to further embodiments of the invention. Exemplary benzimidazoles include thiabendazole, mebendazole, fenbendazole, cambendazole, oxibendazole, flubendazole, oxfendazole, and albendazole.

Pro-benzimidazoles (prodrugs that yield benzimidazoles after biotransformation) can also be used as therapeutic agents for the treatment of bladder disorders according to further embodiments of the invention. Exemplary pro-benzimidazoles include Febantel, which yields febendazole and oxfendazole following injection. See, for example, Armour, “Modern Anthelmintics for Farm Animals”, Chapter 10 in Pharmacological Basis of Large Animal Medicine, eds. J. A. Bogan, P. Lees, and A. T. Yoxall, Blackwell Scientific Publications, Boston, (1983). Other exemplary pro-benzimidazoles which can be used as therapeutic agents for the treatment of bladder disorders according to the invention include albendazole, fenbendzole, and oxfendazole, which are less soluble derivatives which appear to be more effective because of the increased exposure afforded by the prolonged dissolution in the bladder lumen).

Generally, the bladder enhancing agent alone or in combination with one or more therapeutic agents are administered as compositions in a pharmaceutically acceptable excipient, including, but not limited to, saline solutions, suitable buffers, preservatives, stabilizers, and the like. The pharmaceutically acceptable excipients used are those routinely employed by those of skill in the art to deliver the subject agent(s). The excipients used in any given formulation will be compatible with the bladder enhancing agent and with the one or more therapeutic agents such that there is no interaction which would impair the performance of any of the components of the composition. The compositions are suitable for systemic administration to individuals in unit dosage forms, sterile solutions or suspensions, oil in water or water in oil emulsions and the like. Formulations for drug delivery are known in the art, many examples of which are described in Remington's Pharmaceutical Sciences, 18^{th} Edition, Mack Publishing (1990).

Experimental

The following examples are provided for illustrative purposes.

### EXAMPLE 1

Pretreatment and Intravesical Instillation of Compounds in Mice

Female Balb/c mice were used due to the ease of urethral cannulation and intravesical instillation. All animals were housed according to institutional regulations for experimental animals. Balb/c mice (20 gm body weight) were anesthetized with Isoflurane, and a 24-gauge catheter was introduced through the urethra into the bladder. The residual urine was emptied and the bladder was flushed three times with 100 μL of PBS employing a 25-gauge needle inserted into the external end of the catheter. After washing, bladder pretreatment was performed employing the various test reagents as follows: the pretreatment reagent (DDM) was diluted to the desired concentration in PBS, and the bladder was quickly rinsed twice with 100 μL each of the reagent. After the rinsing steps, 100 μL of the pretreatment reagent was introduced via the catheter and retained in the bladder for 5 minutes, followed by an additional quick rinse with the pretreatment reagent. The bladder was then washed three times with 100 μL each of PBS. Each group of mice received one of the following compounds: Group 1—PBS control, Group 2—Oregon Green 488 Paclitaxel (5 μg), Group 3—FITC-LC-TAT (5 μg), Group 4—0.1% Sodium Fluorescein.

70 μL of the solution was intravesically instilled in the bladder and retained for 15 min after which the bladder was washed 3 times with 100 μL each of PBS. Bladders were then harvested and flash frozen for the preparation of cyrosections and microscopy. Blood was withdrawn by cardiac puncture from mice in group 4. The serum was deglucuronidated by incubating 100 μL of the aliquot in 200 μL of 0.2 M acetate buffer (pH=5.4) and 100 μL of β-Glucuronidase (7000 U/ml; from concentrated Calbiochem #B56959). Triplicate samples were adjusted to 2.00 mL with 0.2 M carbonate buffer (pH=9.34) and spectrophotometric emission was measured at 538 nm following excitation at 485 nm. Control and standard NaF solutions were used to determine the concentration of NaF in each plasma sample.

### EXAMPLE 2

Oregon Green 488 Paclitaxel Uptake in Bladder

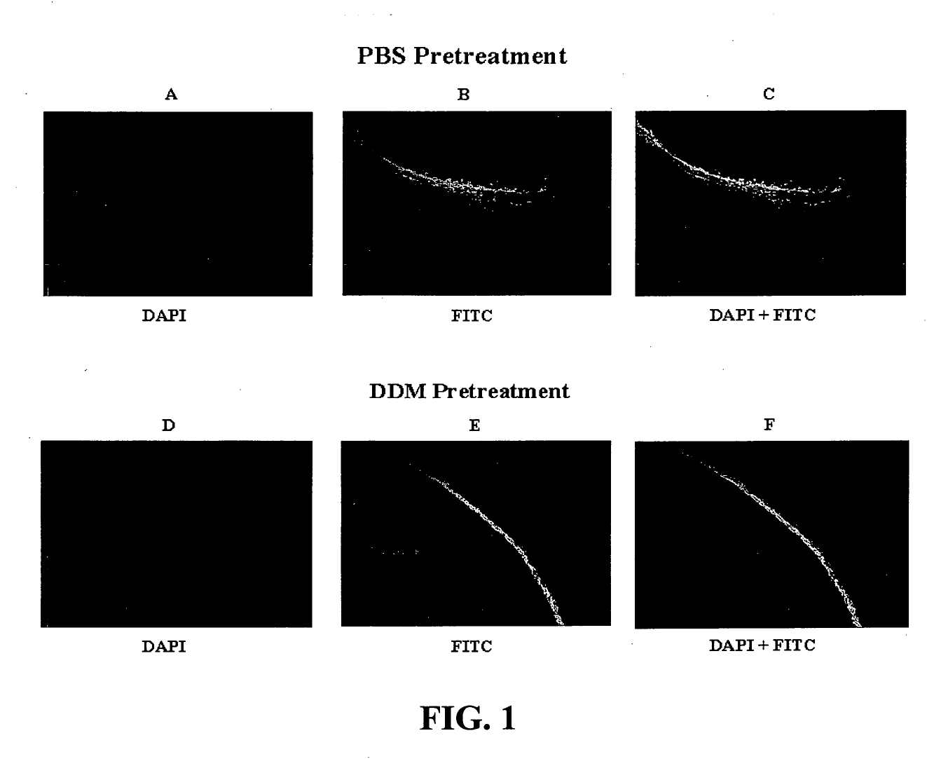

On examination of the histological sections of the bladder that had been pretreated with PBS followed by the intravesical retention of Paclitaxel, it was observed that there were very small patchy areas of non-specifically bound drug on the luminal surface of the bladder (FIGS. 1A-1C). Most of the bladder surface was devoid of any fluorescence that indicated the absence of any entry of the drug into the bladder tissue. In contrast, following DDM pretreatment there was significant uptake of Paclitaxel as evident by a strong fluorescence that was observed in the lamina propria region of the bladder (FIGS. 1D-1F). Within the 15 min. duration of drug retention in the bladder, there was rapid movement of the drug through the urothelial layer and lamina propria of the bladder wall into the muscular layer. Even within the 15 min. time period for which the chemotherapeutic agent was retained in the bladder, significant uptake of the drug was observed to have occurred following DDM pretreatment in contrast to the absence of any uptake in the PBS pretreated bladders. Similar to our earlier observation with Adenovirus infection of the bladder epithelium, DDM pretreatment seems to have led to a transient break in the barrier function that has allowed for rapid uptake of the chemotherapeutic agent within a very time period.

### EXAMPLE 3

FITC-LC-TAT Peptide Uptake in Bladder

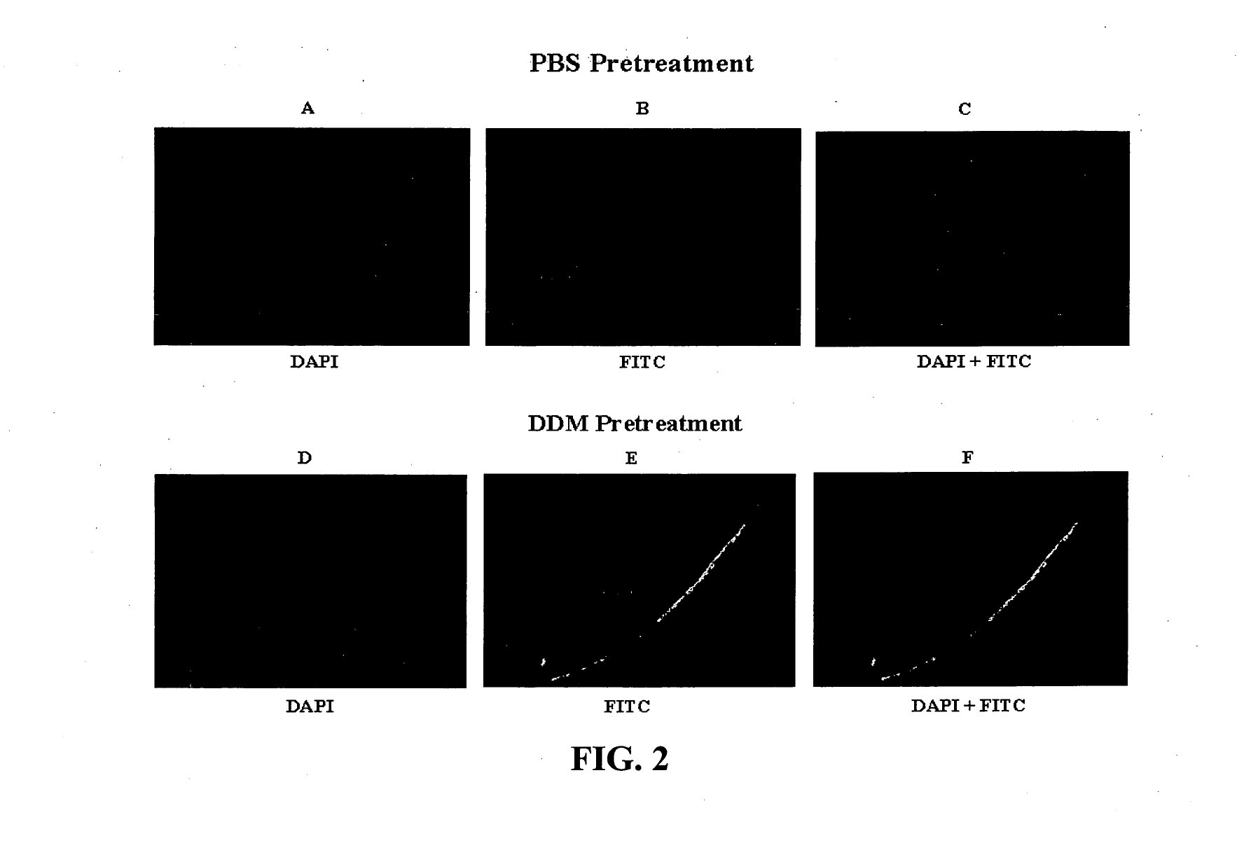

Membrane permeation peptides, such as TAT basic domain, have emerged as useful agents with potential utility in therapeutic delivery and diagnostic imaging [Prochiantz, A. (2000). Curr. Opin. Cell Biol. 12: 400-406; Schwarze, S et al., (1999) Science, 285, 1569-1572]. An FITC tagged TAT peptide was used as a model system to demonstrate the potential effects of DDM pretreatment on the entry of the peptide into the epithelial cells on the luminal surface of the bladder wall. Following pretreatment of the bladder with either PBS or 0.1% solution of DDM, a 5 μg solution of FITC-LC-TAT solution was instilled in the lumen of the bladder and retained for 15 min. After 3× washing of the bladder with PBS, the bladder was harvested and processed for histological examination as mentioned in the Methods. On analysis of the histological sections of the bladder under a microscope, no fluorescent signal could be observed in the PBS pre-treated bladder (FIGS. 2A-2C) whereas a distinct signal could be observed specifically in the epithelial layer on the luminal surface of the bladder treated with DDM (FIGS. 2D-2F). Earlier in vitro studies in certain well-differentiated epithelial cells that form tight junctions demonstrated complete permeation barrier to TAT-peptides [Violini, S et al., (2002) Biochemistry. 41: 12652-12661]. However treatment of plasma membrane permeabilizing agents led to translocation of the peptide into the cells. Our earlier EM studies on DDM pretreated bladder has shown that the pretreatment results not only in the distinct changes in the GAG permeability but also leads to the opening up of the tight junctions in the epithelial cell layer. The transiently opened tight junctions following DDM pretreatment may be allowing for the rapid uptake of the fluorescein conjugated TAT peptide by the epithelial layer.

### EXAMPLE 4

Uptake of Sodium Fluorescein by Bladder Following DDM Pretreatment

Our earlier studies have demonstrated that DDM pretreatment of the bladder leads to a transient break in the permeability barrier thus allowing for effective transduction of the urothelium by adenovirus (See U.S. patent application Ser. Nos. 10/327,869 and 10/743,813). Fluorescein has been used to measure alterations in bladder permeability after bladder mucosal injury in mice [Eichel, L. et al., (2001) Urology. 58: 113-118]. Intravesically instilled sodium fluorescein uptake was utilized as a model compound to test the utility of DDM pretreatment for therapeutic applications. Mouse bladders were pretreated for 5 min with a 0.1% solution of DDM, following instillation with a 0.1% solution of sodium fluorescein into the bladder and retention for 15 min. Following 3× wash of the bladder with PBS, blood was removed from the mice via cardiac puncture and the serum was analyzed for systemic distribution of intravesically instilled sodium fluorescein as described above. DDM pretreatment led to a 5-10× increase in the circulating levels of fluroescein as compared to PBS pretreatment. The rapid systemic biodistribution of a compound following DDM pretreatment of the bladder may serve as a valuable strategy in the management of bladder disease.

The results described herein show that a break in the bladder permeability barrier follows DDM pretreatment, which provides a means for rapid absorption of small molecules such as chemotherapeutic agents, peptides and other chemicals for therapeutic applications.

While the foregoing specification teaches the principles of the present invention, with examples provided for the purpose of illustration, it will be appreciated by one skilled in the art from reading this disclosure that various changes in form and detail can be made without departing from the true scope of the invention.

All literature and patent references cited above are hereby expressly incorporated by reference herein in their entirety.

1. A method for treating a bladder disorder comprising:
contacting, through instillation, a luminal surface of the bladder with a pretreatment composition comprising a bladder uptake enhancing agent; and
subsequently contacting, through instillation, the luminal surface of the bladder with a composition comprising a therapeutic agent for the treatment of the bladder disorder;
wherein the bladder uptake enhancing agent is a mono-, di-, or poly-saccharide having a lipophilic substituent.

2. The method of claim 1, wherein the bladder disorder is bladder cancer.

3. The method of claim 1, wherein the bladder uptake enhancing agent is represented by the following general formula (I) or the following general formula (II): 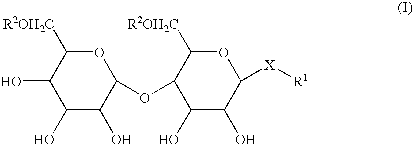 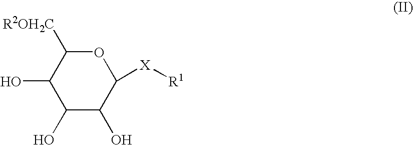
wherein X is a sulfur or oxygen atom, R1 is an alkyl group and each R2 is independently hydrogen or a moiety represented by the following formula: 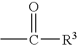
wherein R3 is an alkyl group.

4. The method of claim 1, wherein the pretreatment composition further comprises an oxidizing agent.

5. The method of claim 1, wherein the therapeutic agent is a chemotherapeutic agent, a cytokine, a small molecule drug, a natural product, a radionuclide, or a protein.

6. The method of claim 1, wherein the bladder uptake enhancing agent is n-dodecyl-β-D-maltoside (DDM).

7. A method of treating a bladder disorder comprising:
contacting, through instillation, a luminal surface of the bladder with a pretreatment composition comprising a bladder uptake enhancing agent having a structure represented by the chemical formula: 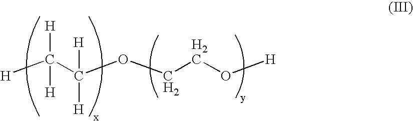
wherein x and y are positive integers;
and subsequently contacting, through instillation, the luminal surface of the bladder with a composition comprising a therapeutic agent for the treatment of the bladder disorder.

8. The method of claim 7, wherein the bladder disorder is bladder cancer.

9. The method of claim 7, wherein x is 6 and y is 8-10 and the pretreatment composition comprises about 0.02 to about 0.05 wt % of the bladder uptake enhancing agent.

10. The method of claim 7, wherein the therapeutic agent is a chemotherapeutic agent, a cytokine, a small molecule drug, a natural product, a radionuclide, or a protein.

11. A method of treating a bladder disorder comprising:
contacting, through instillation, a luminal surface of the bladder with a pretreatment composition comprising a bladder uptake enhancing agent having a structure represented by the following general formula (IV) or the following general formula (V): 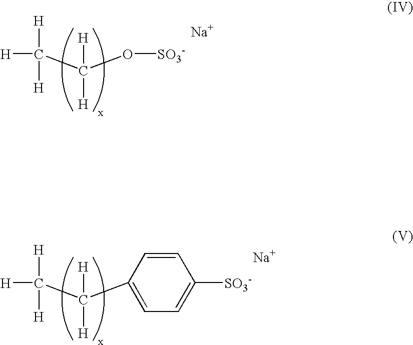
wherein x is a positive integer; and
subsequently contacting, through instillation, the luminal surface of the bladder with a composition comprising a therapeutic agent for the treatment of the bladder disorder.

12. The method of claim 11, wherein the bladder disorder is bladder cancer.

13. The method of claim 11, wherein the therapeutic agent is a chemotherapeutic agent, a cytokine, a synthetic small molecule drug, a natural product, a radionuclide, or a protein.

14. A composition comprising:
a bladder uptake enhancing agent; and a therapeutic agent for the treatment of a bladder disorder; wherein the bladder uptake enhancing agent is a non-immunogenic poly- mono- or di-saccharide having a lipophilic substituent.

15. The composition of claim 14, wherein the bladder disorder is bladder cancer.

16. The composition of claim 14, wherein the bladder uptake enhancing agent is a compound having the following general formula (I) or the following general formula (II): 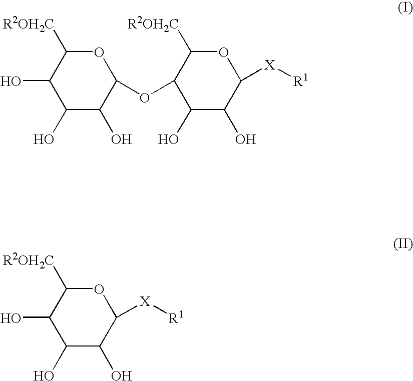
wherein X is a sulfur or oxygen atom, R1 is an alkyl group and each R2 is independently hydrogen or a moiety represented by: 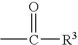
wherein R3 is an alkyl group.

17. The composition of claim 14, wherein the bladder uptake enhancing agent is n-dodecyl-β-D-maltoside (DDM).

18. The composition of claim 14, wherein the therapeutic agent is a chemotherapeutic agent, a cytokine, a synthetic small molecule drug, a natural product, a radionuclide, or a protein.

19. A method for treating a bladder disorder comprising contacting, through instillation, a luminal surface of the bladder with a composition as set forth in claims 14 and 16 to 18.

20. (canceled)

21. The method of claim 19, wherein the bladder disorder is bladder cancer.

22. A composition comprising:
a bladder uptake enhancing agent; and
a therapeutic agent for the treatment of a bladder disorder;
wherein the bladder uptake enhancing agent has a structure represented by the chemical formula: 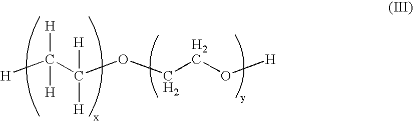
wherein x and y are positive integers.

23. The composition of claim 22, wherein x is 6 and y is 8-10 and wherein the composition comprises about 0.02 to about 0.05 wt % of the bladder uptake enhancing agent.

24. The composition of claim 22, wherein the therapeutic agent is a chemotherapeutic agent, a cytokine, a synthetic small molecule drug, a natural product, a radionuclide, or a protein.

25. A method for treating a bladder disorder comprising contacting, through instillation, a luminal surface of the bladder with a composition as set forth in any one of claims 22 to 24.

26. The method of claim 25, wherein the bladder disorder is bladder cancer.

27. A composition comprising:
a bladder uptake enhancing agent; and
a therapeutic agent for the treatment of a bladder disorder;
wherein the bladder uptake enhancing agent has a structure represented by the following general formula (IV) or the following general formula (V): 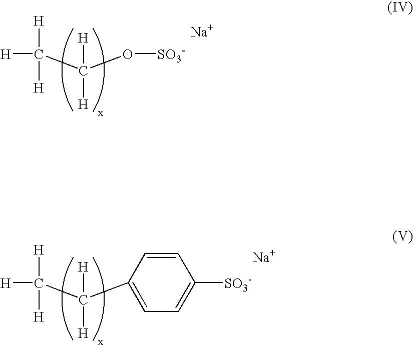
wherein x is a positive integer.

28. The composition of claim 27, wherein the therapeutic agent is a chemotherapeutic agent, a cytokine, a synthetic small molecule drug, a natural product, a radionuclide, or a protein.

29. A method for treating a bladder disorder comprising contacting, through instillation, a luminal surface of the bladder with a composition as set forth in claims 27 or 28.

30. The method of claim 29, wherein the bladder disorder is bladder cancer.

31. The composition of claim 14, wherein the composition further comprises an oxidizing agent.
